(19) United States
(12) Patent Application Publication
(10) Pub. No.: US 2025/0020624 A1
(43) Pub. Date: Jan. 16, 2025

(54) SYSTEMS AND METHODS FOR DETERMINING WATER QUALITY PARAMETERS

(71) Applicant: Flying Gybe Inc., Corvallis, OR (US)

(72) Inventors: Nicholas TUFILLARO, Corvallis, OR (US); Philipp Michael Maximilan GRÖTSCH, Berlin (DE); Ivan LALOVIC, Portland, OR (US)

(21) Appl. No.: 18/771,734

(22) Filed: Jul. 12, 2024

Related U.S. Application Data
(60) Provisional application No. 63/513,179, filed on Jul. 12, 2023.

(51) Int. Cl. G01N 33/18; G06F 17/18

(52) U.S. Cl. CPC G01N 33/188; G01N 33/1886; G06F 17/18

(57) ABSTRACT

Target water quality parameters may be estimated based on measurements of surrogate water quality parameters, which may be easier to measure than the target parameters. Systems and methods in accordance with aspects of the present teachings may include determining correlations between surrogate parameters and a target parameter using a training sample of data, and using the correlations to estimate values of the target parameter corresponding to out-of-sample measurements of the surrogate parameters. In some examples, determining the correlation between the surrogate parameters and the target parameter includes developing a nonlinear surrogate model that can be described as an almost piecewise linear model.

### CROSS-REFERENCES

The following applications and materials are incorporated herein, in their entireties, for all purposes: U.S. Provisional Patent Application Ser. No. 63/513,179, filed Jul. 12, 2023; “A manifold learning perspective on surrogate modeling of nitrate concentration in the Kansas River,” Water Practice & Technology Vol 19 No 4, 1148 (2024).

### FIELD

This disclosure relates to systems and methods for determining parameters related to water quality, including predicting values of a target parameter based on known values of surrogate parameters.

### INTRODUCTION

Estimation of water quality parameters is important for many applications. Water quality parameters can indicate a variety of information about the water in question, including the presence of toxins, acceptable taste or odor of drinking water, and more. Accordingly, various water quality parameters are estimated in various different settings. For example, government regulations may require monitoring water for potentially hazardous substances such as lead. As another example, when a water source is intended to provide drinkable water for municipal customers, turbidity and other parameters associated with drinking water quality may be estimated.

Some water quality parameters can be monitored with in situ, relatively low cost sensors in the natural environment (e.g., at a river or other body of water), or in a water quality treatment plant. Examples of such parameters include, in at least some cases, turbidity, temperature, conductivity (a proxy for salinity), and some organics such as chlorophyll-a. However, many important water quality parameters are difficult or costly to measure, such as concentrations of certain substances such as nutrients, nitrates, and phosphorus. According to some conventional methods, these parameters can be estimated using surrogate models, which quantitatively correlate parameters that are easier to measure with parameters that are difficult to measure. However, conventional surrogate model methods use mathematically linear models that are computationally manageable, but sometimes not as accurate as desired. Better solutions are needed for estimating water quality parameters that are difficult to measure directly.

### SUMMARY

The present disclosure provides systems, apparatuses, and methods relating to determining water quality parameters.

In some examples, a method for predicting a value of a target parameter based on measurements of surrogate parameters comprises: measuring a first dataset comprising time series data for a plurality of parameters including at least a first surrogate parameter, a second surrogate parameter, a third surrogate parameter, and the target parameter; identifying a plurality of regions in a multidimensional space corresponding to clusters of points of the first dataset in the multidimensional space, wherein the multidimensional space has dimensions corresponding to at least a subset of the plurality of parameters; determining a nonlinear manifold comprising a plurality of modeling functions multiplied by corresponding domain indicator functions, wherein each modeling function models the cluster of points of the first dataset corresponding to a respective one of the regions, and each domain indicator function is defined to be close to one in the region associated with the corresponding modeling function, and close to zero in all of the other regions; measuring a second dataset comprising at least one value of the first surrogate parameter, at least one value of the second surrogate parameter, and at least one value of the third surrogate parameter; and predicting a value of the target parameter corresponding to the second dataset by determining a value of the nonlinear manifold corresponding to the at least one value of the first surrogate parameter, the at least one value of the second surrogate parameter, and the at least one value of the third surrogate parameter.

In some examples, a method of estimating a target parameter comprises: obtaining values of a plurality of surrogate parameters; determining a nonlinear surface in a phase space defined by the plurality of surrogate parameters, wherein the nonlinear surface defines values of the target parameter; and predicting a first value of the target parameter by identifying a value of the nonlinear surface at a point in the phase space corresponding to the obtained values of the plurality of surrogate parameters.

In some examples, a data processing system comprises: one or more processors; a memory; and a plurality of instructions stored in the memory and executable by the one or more processors to: receive values of a plurality of surrogate parameters; determine a nonlinear surface in a phase space defined by the plurality of surrogate parameters, wherein the nonlinear surface defines values of a target parameter; and predicting a first value of the target parameter by identifying a value of the nonlinear surface at a point in the phase space corresponding to the received values of the plurality of surrogate parameters.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a plot depicting an illustrative time series data set embedded in a multidimensional space, in accordance with aspects of the present teachings.

FIG. 2 is a plot depicting the data set of FIG. 1 under a natural logarithm transformation, along with a linear response surface computed according to conventional modeling methods.

FIG. 3 is a plot depicting a two-dimensional projection of the data set of FIG. 2.

FIG. 4 is a plot depicting an illustrative nonlinear domain-indicating surface in accordance with aspects of the present teachings.

FIG. 5 is a plot depicting the data set of FIG. 2 and an illustrative nonlinear response surface in accordance with aspects of the present teachings.

FIG. 6 is another plot of the data set and the nonlinear response surface of FIG. 5.

FIG. 7 is a plot comparing measured values of a target parameter to values predicted by the conventional linear response surface of FIG. 2.

FIG. 8 is a plot comparing measured values of the target parameter to values predicted by the nonlinear response surface of FIG. 5.

FIG. 9 is a plot depicting measured and predicted nitrate values plotted against time, the measured nitrate values including values from the data set of FIG. 2 as well as values outside the data set of FIG. 2.

FIG. 10 is a plot depicting an illustrative supplemental model surface in accordance with aspects of the present teachings.

FIG. 11 is a plot depicting measured and predicted nitrate values plotted against time, the predicted values having been predicted using the supplemental model surface of FIG. 10 in accordance with aspects of the present teachings.

FIG. 12 is a flowchart depicting steps of an illustrative method for predicting a value of a target parameter in accordance with aspects of the present teachings.

FIG. 13 is a flowchart depicting steps of an illustrative method for estimating a target parameter in accordance with aspects of the present teachings.

FIG. 14 is a schematic diagram of an illustrative data processing system in accordance with aspects of the present teachings.

### DETAILED DESCRIPTION

Various aspects and examples of systems and methods for water quality parameter estimation are described below and illustrated in the associated drawings. Unless otherwise specified, a system or method in accordance with the present teachings, and/or components thereof, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Processing logic” describes any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

A “controller” or “electronic controller” includes processing logic programmed with instructions to carry out a controlling function with respect to a control element. For example, an electronic controller may be configured to receive an input signal, compare the input signal to a selected control value or setpoint value, and determine an output signal to a control element (e.g., a motor or actuator) to provide corrective action based on the comparison. In another example, an electronic controller may be configured to interface between a host device (e.g., a desktop computer, a mainframe, etc.) and a peripheral device (e.g., a memory device, an input/output device, etc.) to control and/or monitor input and output signals to and from the peripheral device.

Directional terms such as “up,” “down,” “vertical,” “horizontal,” and the like should be understood in the context of the particular object in question. For example, an object may be oriented around defined X, Y, and Z axes. In those examples, the X-Y plane will define horizontal, with up being defined as the positive Z direction and down being defined as the negative Z direction.

“Providing,” in the context of a method, may include receiving, obtaining, purchasing, manufacturing, generating, processing, preprocessing, and/or the like, such that the object or material provided is in a state and configuration for other steps to be carried out.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

In general, systems and methods in accordance with aspects of the present teachings are configured for obtaining information about water quality parameters (referred to as “target” water quality parameters) using information about other water quality parameters (referred to as “surrogate” water quality parameters). The surrogate parameters may, e.g., be more convenient and/or less costly to measure than the target parameters, and/or measurable with higher precision and/or accuracy than the target parameters. Systems and methods in accordance with aspects of the present teachings obtain information about a target water quality parameter using a nonlinear model, which is shown to predict the target water quality parameter more accurately than conventional linear models in at least some cases. Example systems and methods are described below.

### A. Computer Implementation

Aspects of the systems and methods described herein may be embodied as a computer method, computer system, or computer program product. For example, methods for predicting values of target parameters in accordance with aspects of the present teachings may be partially or completely implemented by computer. Accordingly, aspects of the systems and methods described herein may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the systems and methods may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of the systems and methods described herein may be written in one or any combination of programming languages, including an object-oriented programming language (such as Java, C++), conventional procedural programming languages (such as C), and functional programming languages (such as Haskell). Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of systems and methods in accordance with aspects of the present teachings may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the present teachings. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

### B. Illustrative Nonlinear Surrogate Model

This section describes an illustrative nonlinear surrogate model method for obtaining estimates of target water quality parameters. The method includes using a first data set comprising first values of the surrogate parameters and first values of the target parameter to determine a relationship between the surrogate parameters and the target parameter, and using the determined relationship and a second data set comprising second values of the surrogate parameters to estimate corresponding second values of the target parameter. For example, the first data set (AKA the “training data” or “training data set”) may comprise values measured at a first location along a body of water, and the second data set may comprise values measured at a second location along the body of water. Using the surrogate modeling method described herein, values of the target parameter at the second location can be estimated based on the measured values of the surrogate parameters at the second location, without needing to measure the target parameter at the second location. As another example, the first data set may comprise values measured during a first period of time and the second data set may comprise values measured during a second period of time (e.g., later than or earlier than the first period of time).

The method is described below with reference to a non-limiting example in which the surrogate parameters are turbidity, chlorophyll concentration, and discharge, and the target parameter is nitrate concentration. In general, however, the modeling method may be used for other choices of surrogate parameters and/or other choices of target parameter. Furthermore, the modeling method described herein may also be used for contexts other than water quality sensing; in other words, it is contemplated herein that the modeling method is usable in at least some contexts in which the surrogate parameters and/or target parameter are unrelated to water quality. Examples of such contexts may include mechanical or electronics design and analysis (e.g., models for individual electronic or mechanical components or subsystems, and/or other aspects of design or analysis), aerospace engineering, data mining, and/or any other suitable applications.

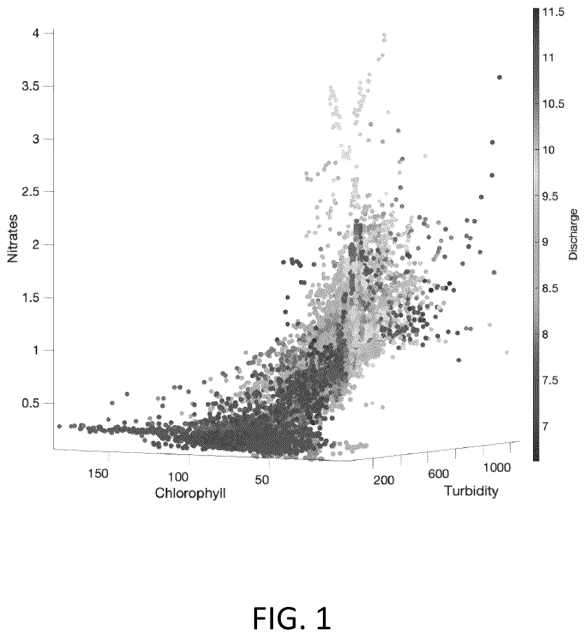

FIG. 1 is a plot depicting an illustrative time series data set embedded in a multidimensional space. The surrogate parameters of turbidity, chlorophyll concentration, and discharge comprise the domain, and the target parameter of nitrate concentration comprises the co-domain (AKA range). More specifically, as discussed herein with reference to this example, “chlorophyll concentration” refers to a concentration of chlorophyll-a, and “nitrate concentration” refers to a concentration of the quantity (NO₃+NO₂), i.e., nitrate plus nitrides.

In the depicted example, the time series data was measured by in situ sensors deployed by the United States Geological Survey (USGS) at the Kansas River at De Soto, Kansas. In general, however, the training data may be obtained in any suitable way (e.g., measured, retrieved from a database of known measurements, estimated, predicted by model(s), and/or obtained in any other suitable manner).

Optionally, the time series data can be processed in a suitable manner before being embedded in the multidimensional space. In the depicted example, the time series data is aggregated into 52 gapless subsets with record lengths varying from 32 (approximately 2 days) to 2916 (approximately 182 days). The total length of the processed data set is 9279 points (approximately 6.3 years). For further description, see “A manifold learning perspective on surrogate modeling of nitrate concentration in the Kansas River,” Water Practice & Technology Vol 19 No 4, 1148 (2024), which is incorporated herein by reference in its entirety for all purposes.

As can be seen in FIG. 1, predictions of the target parameter based on one or more surrogate parameters near the origin of the plot are prone to ambiguity, because there is a high variance in co-domain (target parameter) response for nearby points in the domain. Mathematically, the mapping from surrogate parameters to target parameter near the origin is multivalued (AKA noninjective). Accordingly, in some examples, a logarithmic transformation is applied to the data set to help spread out the data near the origin and to help cause the data to more closely approximate a normal distribution.

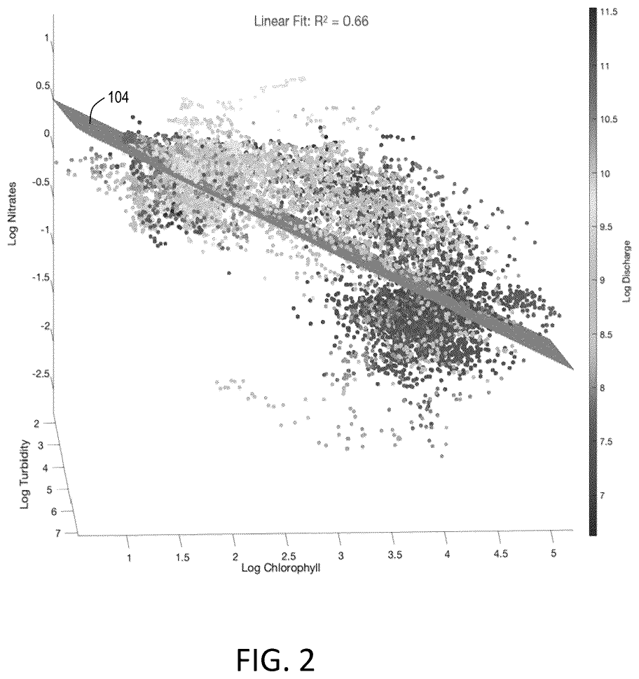

FIG. 2 is a three-dimensional plot depicting the data set of FIG. 1 under a natural logarithm transformation. The plot includes a linear response surface 104, which is computed using conventional linear surrogate modeling methods. Linear response surface 104 may be referred to as a one-to-one or injective map of the input space defined by the surrogate parameters to the output space of the target parameter.

As FIG. 2 shows, the data is skewed into two separate clusters and not normally distributed in the log space. Accordingly, linear response surface 104 fails to describe the plainly nonlinear shape of the data. As a linear surface, surface 104 inherently optimizes the mean of all the data points, and implicitly relies on an assumption that the data is normally distributed. As a result, linear response surface 104 underestimates moderate nitrate values and misses the mean of data in the two clusters of the data set. This helps to show why improved surrogate modeling methods are needed for accurately predicting target water quality parameters.

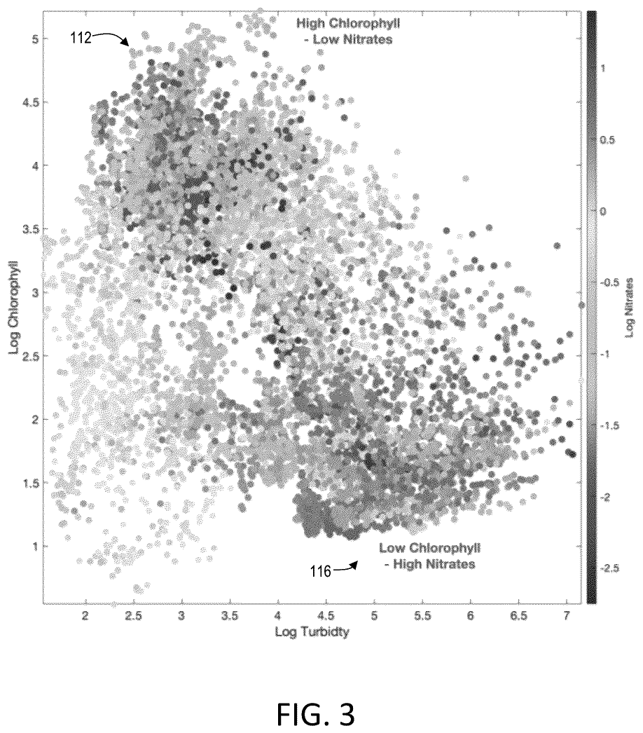

Systems and methods according to aspects of the present teachings involve nonlinear response surfaces configured to better correlate surrogate parameters with a target parameter. Determining a suitable nonlinear response surface includes partitioning the domain into subregions in which different portions of the training data set can be modeled. In this example, the data set comprises two clusters of data, as can be seen in FIG. 2. The two clusters can also be seen in FIG. 3, which is a two-dimensional projection of the log-transformed data set onto a two-dimensional plane defined by two of the surrogate parameters (here, turbidity and chlorophyll concentration). In FIG. 3, color represents the target parameter (nitrate concentration).

In FIG. 3, the two clusters of the data set are disposed at the blue region generally indicated at 112 and the red region generally indicated at 116. Blue region 112 corresponds to high chlorophyll concentration and low nitrate concentration, and red region 116 corresponds to high turbidity and high nitrate concentration. Although the data in this example comprises two clusters, in other examples, the data may comprise more than two clusters. In general, any parameterized submanifold in dimension N is sufficient to model separated clusters in dimension N+1.

Because this training set comprises two clusters, determining a nonlinear response surface includes partitioning the domain into two subregions (AKA domains). This facilitates modeling each cluster using a local model, rather than a single model like conventional linear surface 104, which attempts to model both clusters simultaneously and ends up modeling the data poorly.

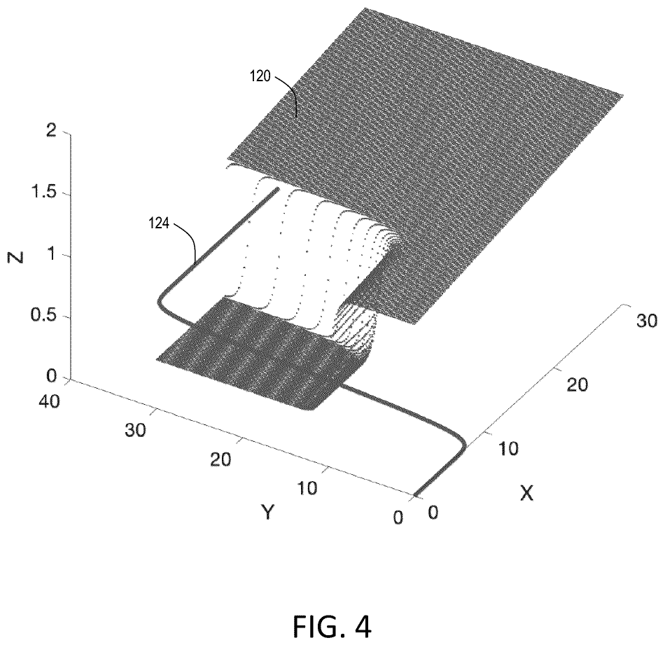

FIG. 4 depicts a three-dimensional plot including a nonlinear domain-indicating surface 120 comprising two domain indicator functions. The first domain indicator function has a value close to 1 at a first domain and a value close to 0 at a second domain; the second domain indicator function has a value close to 0 on the first domain and a value close to 1 on the second domain. Specifically, in the depicted example, surface 120 comprises the following two domain indicator functions:

$${d}_{1} ( x , y ) = \frac{1}{2} ( 1 + tanh ( {ω}_{2} * ( y - \frac{β}{2} ( 1 + tanh ( {ω}_{1} * ( x - {α}_{1} ) ) ) ) - {α}_{2} ) ) {d}_{2} ( x , y ) = \frac{1}{2} ( 1 - tanh ( {ω}_{2} * ( y - \frac{β}{2} ( 1 + tanh ( {ω}_{1} * ( x - {α}_{1} ) ) ) ) - {α}_{2} ) )$$

where the five constants α_{1,2}, ω_{1,2}, and β (referred to herein as domain-indicating constants) are parameters that control the location and steepness of the crossover between the two domains. It is understood that the variables x and y that are used to express the domain-indicating functions here represent dimensions of the phase space defined by the surrogate parameters of the model (e.g., log (turbidity) and log (chlorophyll concentration) in this example). Accordingly, the domain for the function that is the model is selected to be the collection of (x,y) points defined by the sum of the two domain indicator functions.

Domain-indicating surface 120 can be described as two planes continuously connected to one another by a crossover function (in this example, a nonlinear function comprising a hyperbolic tangent). It is desirable that the surface be continuous because this allows the surface to be smooth (i.e., that all derivatives of the surface exist), which allows standard computational toolkits (e.g., for optimization) to be used to implement the model described herein without running into mathematical difficulties associated with lack of continuity. Using a hyperbolic tangent as the crossover function, as in this example, is beneficial because the hyperbolic tangent allows a very quick crossover between the planes. In other examples, however, a different crossover function may be used. In FIG. 4, curve 124 in the X-Y plane marks the separated domains of the planar solution about each cluster.

To create a nonlinear response surface that models the training data set, each domain-indicating function is multiplied by a modeling function that models the data in the respective cluster. In the depicted example, the nonlinear response surface comprises the following two functions:

$$\begin{matrix}{f}_{1} ( x , y ) = ( {a}_{1} x + {b}_{1} y + {c}_{1} ) * {d}_{1} ( x , y ) \\ {f}_{2} ( x , y ) = ( {a}_{2} x + {b}_{2} y + {c}_{2} ) * {d}_{2} ( x , y )\end{matrix}$$

where d₁(x, y) and d₂(x, y) are the domain-indicating functions defined above, and the six constants a_{1,2}, b_{1,2}, and c_{1,2} (referred to herein as modeling constants) define the slope and offset of the modeling functions (a_{1,2}x+b_{1,2}y+c_{1,2}) on each domain. The modeling functions in this example are thus linear approximations to the training data set on each domain; in other examples, however, different modeling functions may be used. Additionally, or alternatively, in other examples different domain-indicating functions may be used. In the example discussed herein, the hyperbolic tangent function is advantageously adjustable to approximate a basic outline of the data in the domain; however, different function(s) may be used in other examples if suitable. The nonlinear response surface defined in this example by f₁(x, y) and f₂(x, y) contains 11 constants (the five domain-indicating constants and six modeling constants) and so by design will not overfit the training data. In other examples, a different suitable number of fitting constants may be used.

The nonlinear response surface defined by f₁(x, y) and f₂(x, y) in accordance with aspects of the present teachings may be referred to as “almost piecewise linear” because the surface is composed of the two linear planes defined by the modeling functions. The word “almost” in “almost piecewise linear” is based on the fact that the nonlinear domain-indicator functions prevent the surface from being truly piecewise linear.

To model a particular training data set, any suitable method(s) may be used to identify values of the domain-indicating constants that define subregions suitable for the training data set (e.g., subregions in which portions of the training data are clustered). Any suitable method(s) may be used to identify values of the modeling constants that adequately model the training data in the corresponding subregion. What it means to adequately model the data may be determined in a given use case based on the needs of the case. In some examples, the modeling constants are selected such that the modeling function, when multiplied by the domain-indicating function, passes through the local mean of the training data in that subregion.

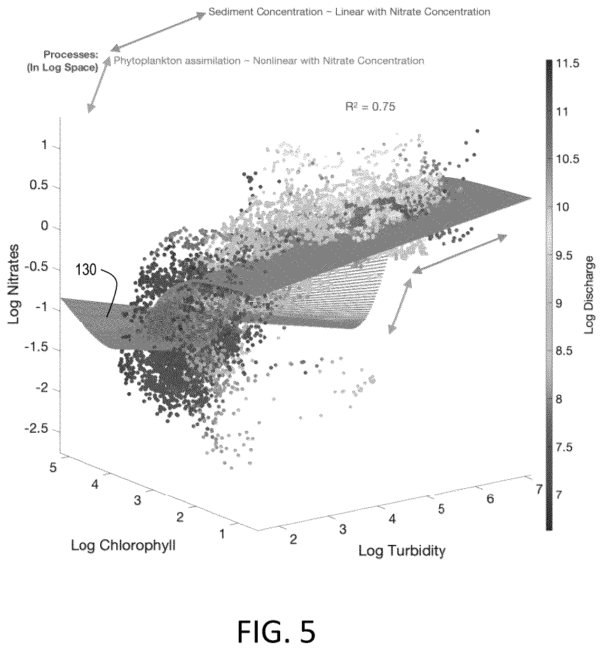

FIG. 5 depicts a three-dimensional plot of the training data set and an illustrative nonlinear response surface 130. Surface 130 comprises the two functions f_{1,2} above, with the following values for the domain-indicating and modeling constants: β=0.5961, α1=3.8200, α2=7.7535, ω1=4.0395, ω2=2.3029, a1=−0.0131, b1=0.2894, c1=−2.5829, a2=0.0922, b2=−0.1125, c2=0.0985. In this example, the domain-indicating constants and the modeling constants were obtained by nonlinear regression. In other examples, however, one or more other methods may additionally or alternatively be used to obtain the domain-indicating constants and/or the modeling constants.

In examples in which the domain-indicating constants and modeling constants are obtained by optimization methods (e.g., nonlinear optimization), the initial values of the constants may be selected in any suitable manner. In some examples, initial values are selected by estimating an initial set of constants that yields a hyperbolic tangent curve separating the two clusters, and an approximate mean value for the response surface in each cluster.

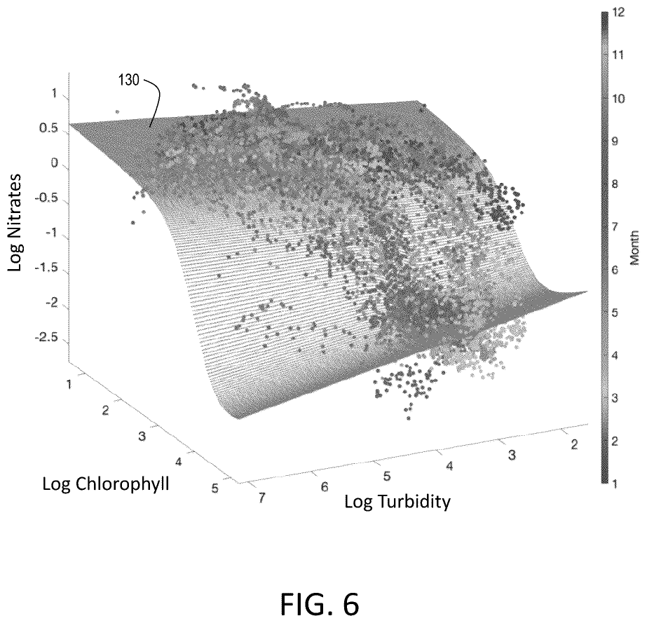

FIG. 6 depicts another three-dimensional plot of the training data set and nonlinear response surface 130. In FIG. 6, the color of the data points indicates a time of the year rather than discharge. The data and surface 130 are also shown from a different point of view than in FIG. 5 (that is, FIG. 6 is rotated relative to FIG. 5).

Nonlinear response surface 130 is a significantly better model of the training data set than linear surface 104 of FIG. 2. Two metrics for evaluating model quality are briefly discussed here. First, the coefficient of determination R² indicates how much of the variance in the dependent variable (i.e., the target parameter) is explained by the independent variables (i.e., the surrogate parameters). A value of R²=0 would indicate that the model is no better than using the mean value for estimation; a value of R²=1 would indicate perfect correlation. For conventional linear surface 104, R²=0.66, and for nonlinear surface 130, R²=0.75. The improved coefficient of determination indicates that the model of the present teachings is a significant improvement over the conventional model.

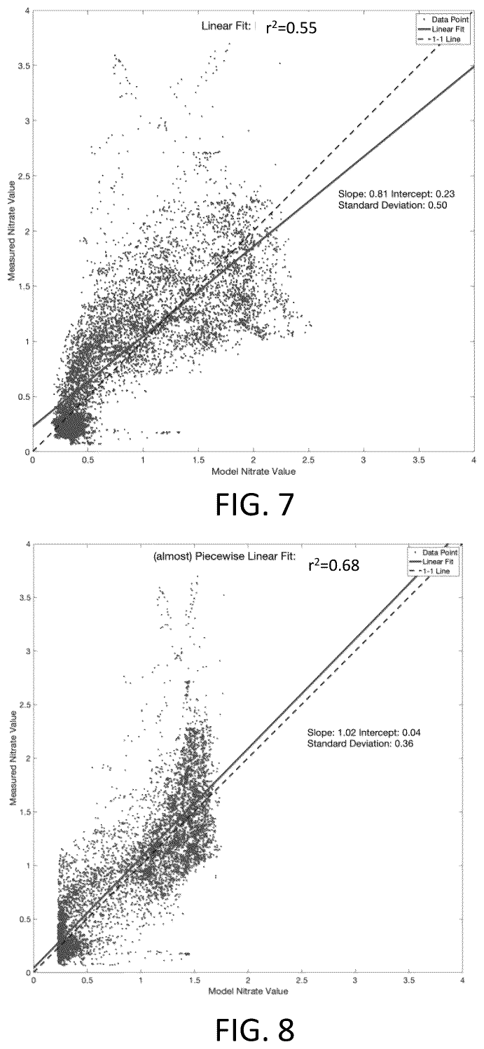

Second, the quality of the conventional model and the quality of the model described herein can be compared by a linear goodness-of-fit metric r² between the measured target parameter and the target parameter predicted by each model. FIG. 7 depicts a two-dimensional plot in which the measured value of the target parameter (nitrate concentration) is plotted against the value predicted by the conventional linear model of surface 104. FIG. 8 depicts a two-dimensional plot in which the measured value of the target parameter is plotted against the value predicted by the almost piecewise linear model of surface 130. Each plot also includes a line showing the best fit between the modeled values and the measured values, as well as a one-to-one line in which the modeled value (horizontal axis) exactly matches the measured value (vertical axis). Notably, the fit for the almost piecewise linear model is very close to the one-to-one line, indicating that the model predicts the measured values very well. The fit for the conventional linear model is not nearly as close to the one-to-one line. The goodness-of-fit metric is r²=0.55 for the conventional model but is r²=0.68 for the model of the present teachings, a significant improvement.

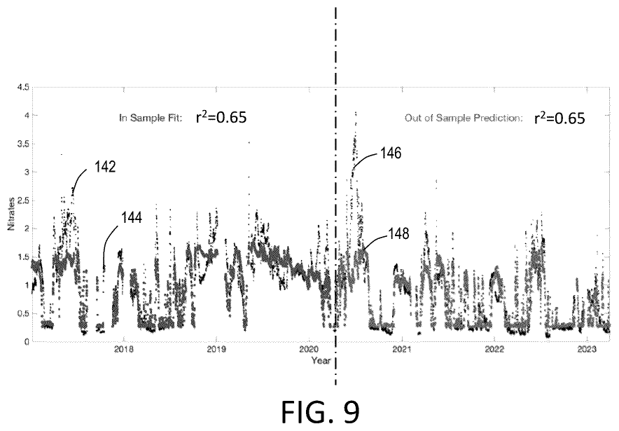

A nonlinear response surface such as surface 130 can be used to predict a value of the target parameter corresponding to out-of-sample values of the surrogate parameters (i.e., data outside the training data set). FIG. 9 depicts nitrate values plotted against time. Specifically, the left portion of the plot of FIG. 9 (i.e., left of the vertical dash-dot line, corresponding to time up until early 2020) includes measured nitrate values from the training data set (the black points, indicated by 142) and predicted nitrate values predicted by the almost piecewise linear model of surface 130 (the blue points, indicated by 144). The right portion of the plot (i.e., right of the vertical dash-dot line, corresponding to time after early 2020) includes measured nitrate values that were not in the training data set (the black points, indicated by 146) and predicted nitrate values (the red points, indicated by 148) that were predicted by the almost piecewise linear model of surface 130 based on measured values of the surrogate parameters corresponding to the measured nitrate values. The overall similarity in appearance of the in-sample and out-of-sample portions of FIG. 9 shows that the model performs well on out-of-sample data. Additionally, the goodness-of-fit parameter is r²=0.65 for both the in-sample and the out-of-sample predictions, which is another indication that the model performs well on both in-sample and out-of-sample data (though it is coincidental that the goodness-of-fit value is identical for the two sets of data, rather than simply close).

In FIG. 9, the model of surface 130 is used to predict nitrate values corresponding to surrogate parameter measurements for which actual nitrate measurements are also available; this is done in order to compare the predictions of the model to the actual nitrate measurements so as to assess the performance of the model. As described elsewhere herein, the model of the present teachings can be used to predict nitrate values corresponding to surrogate parameter measurements for which no actual nitrate measurements are available. This allows nitrate concentration to be estimated in situations where it is not feasible to measure it. Additionally, or alternatively, predictions of the present model may be used to confirm tentative measurements of nitrate concentration, to calibrate a measurement system, and/or used in any other suitable manner.

The nonlinear response surface described above, of which surface 130 is an example, is smooth in the sense that all its derivatives exist. Accordingly, the nonlinear response surface can be described as a manifold. Advantageously, constructing the manifold as described herein leads to a manifold that is numerically stiff.

Optionally, if desired, one or more additional processes can be used to supplement, and/or replace portions of, the predictions of the almost piecewise linear model. This may be useful in situations where it can be determined (or guessed) that the almost piecewise linear model is less accurate in certain regimes than in others. Any suitable supplemental model may be used. For example, a supplemental model may include predictions based on additional variables. A supplemental model may include a probabilistic model such as a Gaussian Process model or a Monte Carlo estimation (e.g., a Markov chain Monte Carlo estimation), and/or any other suitable model, such as the model described below.

As a nonlimiting, illustrative example, FIG. 9 shows that the predictions of the almost piecewise linear model are less accurate when the true nitrate concentration is extreme (e.g., around the middle of the year 2020 in FIG. 9). This may indicate that the linear modeling function has limited accuracy in predicting nitrate concentrations in the high-nitrate cluster (region 116 in FIG. 3). Accordingly, in this example, a supplemental model is used to predict nitrate values in the high-nitrate cluster. The high-nitrate cluster is bounded by 4<Log (Turbidity)<6.6 and 1.2<Log (Chlorophyll concentration)<2.5.

In this example, the supplemental model comprises augmenting nonlinear response surface 130 in the high nitrate cluster by computing a mean value of nitrate concentration as a function of the mean value of discharge and seasonality. Seasonality is expressed by the following function:

$$s ( N ) = ( 1 + sin ( ( \frac{2 π N}{365} ) + \frac{3 π}{2} ) ) / 2$$

where N represents a day of the year (e.g., with N=1 corresponding to January 1). The seasonality function s(N) provides an approximate proxy for seasonal variations of temperature, sunlight, and seasonal fertilizer applications.

Accordingly, on the high-nitrate cluster, the mean value predicted by the almost piecewise linear model is subtracted from the actual predicted value at each point on surface 130. The discharge and seasonality variables are divided into n×n bins; in this example, n=7. In each bin, the mean values of the input and response variables are computed. Accordingly, the domain for the supplemental functional model includes discharge and seasonality (s(N)), and the range is the target parameter, nitrate concentration. The binning yields a cell map. The input and output spaces are broken up into bins, which allows the average value in each input bin to be mapped to the average value in each output bin. This method of averaging helps to smooth out the supplemental functional model without being as rough as, e.g., simply taking an average over the entire input or output variable(s). The result is a set of points of estimated average nitrate values in the high nitrate cluster as a function of the local mean values for discharge and season.

The scattered point data of nitrate values is then used to estimate a surface (referred to herein as the supplemental model surface) using radial basis functions (RBF). Because the seasonality domain is periodic, the seasonality boundaries and the discharge boundaries are pinned to the mean values predicted by the almost piecewise linear model.

Estimating the supplemental model surface includes determining fitting parameters for the radial basis functions. In this example, the radial basis functions are 2-dimensional Gaussian radial basis functions, which have a tuning parameter σ that regularizes the estimated solution (that is, controls the smoothness of the estimated solution). The estimation of the surface using radial basis functions is sensitive to parameters including the regularization tuning parameter σ, the specified boundary conditions, the binning choice (n), and others. Accordingly, the fitting parameters are selected carefully so as to produce a surface that reasonably models the data.

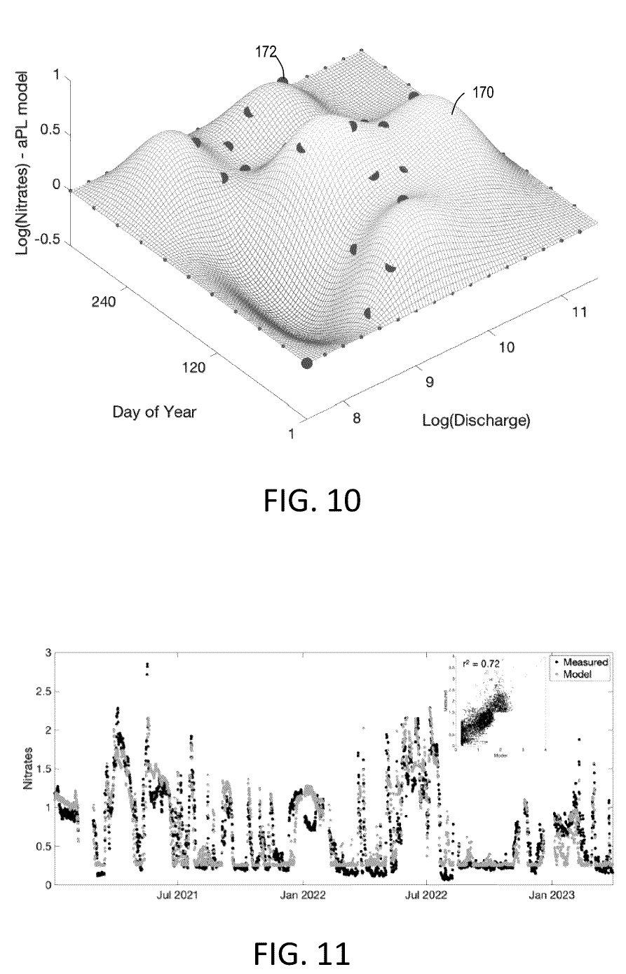

FIG. 10 depicts a three-dimensional plot including an example estimated supplemental model surface 170 configured to predict nitrate values in the high-nitrate cluster. Surface 170 is computed using radial basis functions as described above. Data points 172 are averages over the original data set within the high-nitrate cluster (i.e., region 116 in FIG. 3).

FIG. 11 depicts a plot of measured nitrate value and nitrate value predicted by the supplemental model for a subset of out-of-sample time. The supplemental model shows better agreement with the measured data compared to the almost piecewise linear model for “pulses” of high nitrate concentration values. The goodness-of-fit for the supplemental model is r²=0.72, a noticeable improvement over the value of r²=0.65 achieved by the almost piecewise linear model. Accordingly, augmenting the almost piecewise linear model with a supplemental model, such as the illustrative radial basis function model described above, can lead to improved predictions.

### C. Illustrative Method for Predicting a Value of a Target Parameter

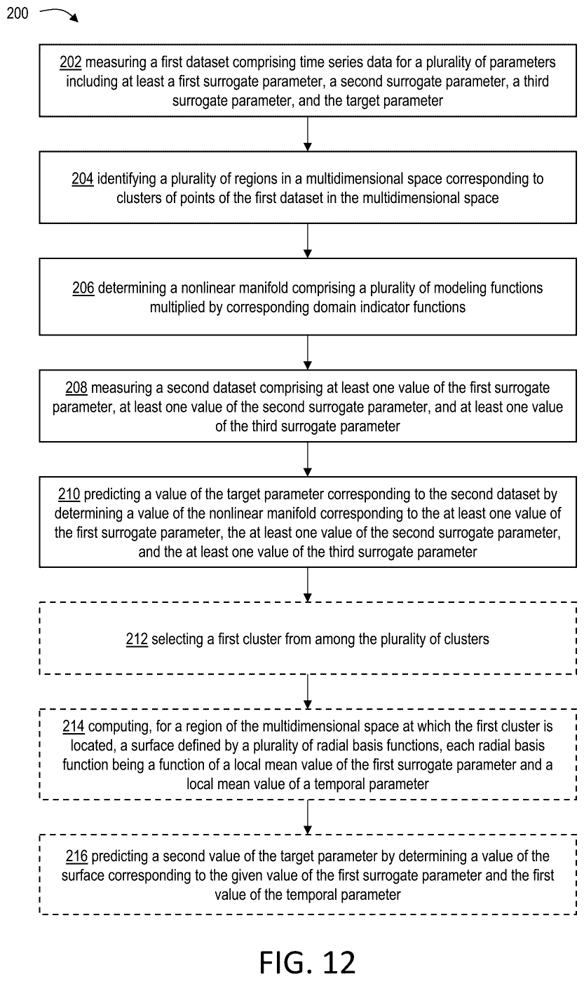

This section describes steps of an illustrative method 200 for predicting a value of a target parameter based on measurements of surrogate parameters; see FIG. 12. Aspects of modeling methods described above may be utilized in the method steps described below.

FIG. 12 is a flowchart illustrating steps performed in method 200, and may not recite the complete process or all steps of the method. Although various steps of method 200 are described below and depicted in FIG. 12, the steps need not necessarily all be performed, and in some cases may be performed simultaneously or in a different order than the order shown.

At step 202, method 200 includes measuring a first dataset comprising time series data for a plurality of parameters including at least a first surrogate parameter, a second surrogate parameter, a third surrogate parameter, and the target parameter. In some examples, the target parameter is a water quality parameter, and the first, second, and third surrogate parameters are discharge rate, turbidity, and chlorophyll respectively. In some examples, more than three surrogate parameters are used.

At step 204, method 200 includes identifying a plurality of regions in a multidimensional space corresponding to clusters of points of the first dataset, wherein the multidimensional space has dimensions corresponding to at least a subset of the plurality of parameters.

At step 206, method 200 includes determining a nonlinear manifold comprising a plurality of modeling functions multiplied by corresponding domain indicator functions. Each modeling function models the cluster of points of the first dataset corresponding to a respective one of the regions, and each domain indicator function is defined to be close to one in that region and close to zero in all other regions.

In some examples, the modeling functions and domain indicator functions are selected such that the nonlinear manifold passes through a local mean of each cluster of points of the first dataset. In some examples, the domain indicator functions comprise hyperbolic tangents. Determining the nonlinear manifold may comprise obtaining a plurality of fitting parameters of the modeling functions and/or domain indicator functions using a suitable fitting method, such as a nonlinear regression. Initial fitting parameter values for the fitting method may be selected in any suitable manner. For example, the initial fitting parameter values may be selected such that each modeling function, when evaluated using its set of initial fitting parameters, has a value equal to a mean value of the target parameter of the cluster of points corresponding to that modeling function.

At step 208, method 200 includes measuring a second dataset comprising at least one value of the first surrogate parameter, at least one value of the second surrogate parameter, and at least one value of the third surrogate parameter.

At step 210, method 200 includes predicting a value of the target parameter corresponding to the second dataset by determining a value of the nonlinear manifold corresponding to the at least one value of the first surrogate parameter, the at least one value of the second surrogate parameter, and the at least one value of the third surrogate parameter.

Optionally, the predictions yielded by the nonlinear manifold may be augmented for one or more clusters. For example, this may be appropriate if the modeling function for a given region of the multidimensional space approximates the target parameter at that region with less accuracy than desired. Accordingly, at step 212, method 200 optionally includes selecting a first cluster from the plurality of clusters for which augmented prediction is desired.

At step 214, method 200 optionally includes computing, for the region of the multidimensional space where the selected first cluster is located, a surface defined by a plurality of radial basis functions. Each radial basis function is a function of a local mean value of the first surrogate parameter and a local mean value of a temporal parameter (e.g., a seasonality parameter). In other examples, different functions may be used to define the surface of step 214.

At step 216, method 200 optionally includes predicting a second value of the target parameter, the second value of the target parameter corresponding to a given value of the first surrogate parameter at a time corresponding to a first value of the temporal parameter, by determining a value of the surface corresponding to the given value of the first surrogate parameter and the first value of the temporal parameter.

### D. Illustrative Method of Estimating a Target Parameter

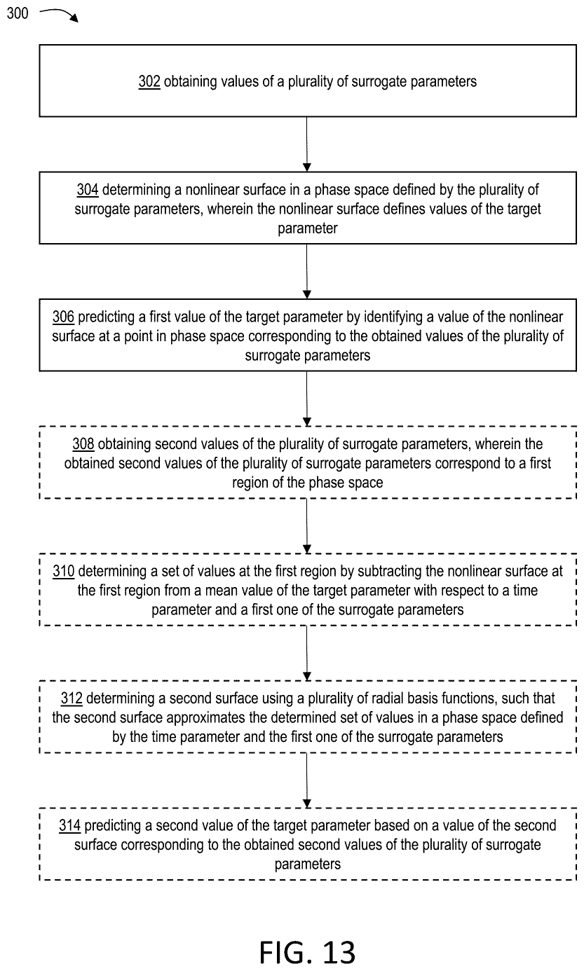

This section describes steps of an illustrative method 300 for estimating a target parameter; see FIG. 13. Aspects of modeling methods described above may be utilized in the method steps described below.

FIG. 13 is a flowchart illustrating steps performed in method 300, and may not recite the complete process or all steps of the method. Although various steps of method 300 are described below and depicted in FIG. 13, the steps need not necessarily all be performed, and in some cases may be performed simultaneously or in a different order than the order shown.

At step 302, method 300 includes obtaining values of a plurality of surrogate parameters.

At step 304, method 300 includes determining a nonlinear surface in a phase space defined by the plurality of surrogate parameters, wherein the nonlinear surface defines values of the target parameter. In some examples, determining the nonlinear surface comprises determining a plurality of local surfaces each configured to model the target parameter in a respective region of the phase space, and joining the plurality of local surfaces together using a plurality of joining functions. The joining functions may be configured to have a nonzero value at a respective one of the regions and a zero value outside that region.

In examples in which step 304 includes determining a plurality of local surfaces, the local surfaces may be determined by embedding a plurality of known data points in the phase space. Each known data point comprises, for a respective point in time, known values of the target parameter and the plurality of surrogate parameters. In such examples, determining the plurality of local surfaces further comprises identifying a plurality of clusters of the known data points in the phase space, and defining respective locations of the plurality of clusters in the phase space to be the regions of the phase space referred to above with respect to step 304. Determining the plurality of local surfaces further comprises selecting, as each one of the local surfaces, a surface that is linear in the corresponding region of the phase space and passes through a mean of the known values of the target parameter in the corresponding region. In some examples, the local surfaces are defined by constants, and determining the plurality of local surfaces includes using a nonlinear regression to obtain values of the constants.

At step 306, method 300 includes predicting a first value of the target parameter by identifying a value of the nonlinear surface at a point in phase space corresponding to the obtained values of the plurality of surrogate parameters obtained at step 302. In some examples, these obtained values correspond to a point in time that is later than any of the times corresponding to the plurality of known data points used (in some examples) at step 304 to determine the nonlinear surface. Described another way, the nonlinear surface may have been determined using data from an earlier point in time, and step 306 may include predicting a value of the target parameter at a later point in time based on surrogate parameter data from the later point in time. Alternatively, or additionally, the nonlinear surface may have been determined using data from a first geographic location, and step 306 may include predicting a value of the target parameter at a different geographic location based on surrogate parameter data from that different geographic location.

At step 308, method 300 optionally includes obtaining second values of the plurality of surrogate parameters, wherein the obtained second values of the plurality of surrogate parameters correspond to a first region of the phase space.

At step 310, method 300 optionally includes determining a set of values at the first region by subtracting the nonlinear surface at the first region from a mean value of the target parameter with respect to a time parameter and a first one of the surrogate parameters. In some examples, the time parameter is a proxy for seasonality; for example, the time parameter may vary sinusoidally with time of year. In some examples, the sinusoidal function is at a maximum approximately halfway through the year (e.g., during Northern Hemisphere summer) and at a minimum at the beginning of the year (e.g., during Northern Hemisphere winter). In this manner, the time parameter can account for seasonal variations in temperature, sunlight, fertilizer application, and/or other suitable parameter(s). The phase and other aspects of the sinusoidal function can be adjusted as appropriate (e.g., to account for a different location on the globe, such as a Southern Hemisphere location, and/or on any other suitable basis).

At step 312, method 300 optionally includes determining a second surface using a plurality of radial basis functions, such that the second surface approximates the set of values determined at step 310 in a phase space defined by the time parameter and the first one of the surrogate parameters.

At step 314, method 300 optionally includes predicting a second value of the target parameter based on a value of the second surface corresponding to the obtained second values of the plurality of surrogate parameters obtained at step 308. In some examples, this value of the second surface is used to augment a prediction of the target value for those surrogate parameter values that was obtained from the nonlinear surface of step 304. For example, the first region of the phase space referred to in steps 308-314 may be a region of phase space for which the nonlinear surface provides less accurate predictions than desired, such that augmenting those predictions using the second surface tends to yield more accurate predictions of target parameter value.

### E. Illustrative Data Processing System

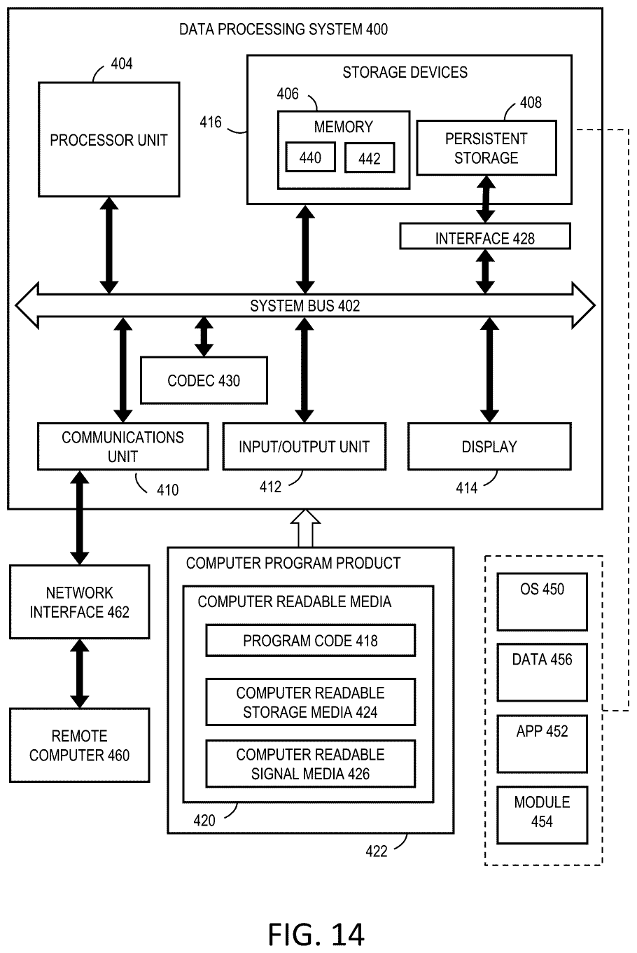

With reference to FIG. 14, this section describes an illustrative data processing system 400 (also referred to as a computer, computing system, and/or computer system) in accordance with aspects of the present teachings. In some examples, devices that are embodiments of data processing system 400 (e.g., smartphones, tablets, personal computers) are used to carry out surrogate modeling methods (or aspects thereof) as described herein. For example, one or more data processing systems may be used to store, receive, transmit, and/or process measurements of surrogate water quality parameters. As another example, one or more data processing systems may be used to compute a nonlinear response surface in accordance with aspects of the present teachings (including, e.g., determining modeling constants and/or domain-indicating constants of the nonlinear response surface). In general, one or more data processing systems may be used to implement the models and methods described in this disclosure.

In this illustrative example, data processing system 400 includes a system bus 402 (also referred to as communications framework). System bus 402 may provide communications between a processor unit 404 (also referred to as a processor or processors), a memory 406, a persistent storage 408, a communications unit 410, an input/output (I/O) unit 412, a codec 430, and/or a display 414. Memory 406, persistent storage 408, communications unit 410, input/output (I/O) unit 412, display 414, and codec 430 are examples of resources that may be accessible by processor unit 404 via system bus 402.

Processor unit 404 serves to run instructions that may be loaded into memory 406. Processor unit 404 may comprise a number of processors, a multi-processor core, and/or a particular type of processor or processors (e.g., a central processing unit (CPU), graphics processing unit (GPU), etc.), depending on the particular implementation. Further, processor unit 404 may be implemented using a number of heterogeneous processor systems in which a main processor is present with secondary processors on a single chip. As another illustrative example, processor unit 404 may be a symmetric multi-processor system containing multiple processors of the same type.

Memory 406 and persistent storage 408 are examples of storage devices 416. A storage device may include any suitable hardware capable of storing information (e.g., digital information), such as data, program code in functional form, and/or other suitable information, either on a temporary basis or a permanent basis.

Storage devices 416 also may be referred to as computer-readable storage devices or computer-readable media. Memory 406 may include a volatile storage memory 440 and a non-volatile memory 442. In some examples, a basic input/output system (BIOS), containing the basic routines to transfer information between elements within the data processing system 400, such as during start-up, may be stored in non-volatile memory 442. Persistent storage 408 may take various forms, depending on the particular implementation.

Persistent storage 408 may contain one or more components or devices. For example, persistent storage 408 may include one or more devices such as a magnetic disk drive (also referred to as a hard disk drive or HDD), solid state disk (SSD), floppy disk drive, tape drive, Jaz drive, Zip drive, flash memory card, memory stick, and/or the like, or any combination of these. One or more of these devices may be removable and/or portable, e.g., a removable hard drive. Persistent storage 408 may include one or more storage media separately or in combination with other storage media, including an optical disk drive such as a compact disk ROM device (CD-ROM), CD recordable drive (CD-R Drive), CD rewritable drive (CD-RW Drive), and/or a digital versatile disk ROM drive (DVD-ROM). To facilitate connection of the persistent storage devices 408 to system bus 402, a removable or non-removable interface is typically used, such as interface 428.

Input/output (I/O) unit 412 allows for input and output of data with other devices that may be connected to data processing system 400 (i.e., input devices and output devices). For example, an input device may include one or more pointing and/or information-input devices such as a keyboard, a mouse, a trackball, stylus, touch pad or touch screen, microphone, joystick, game pad, satellite dish, scanner, TV tuner card, digital camera, digital video camera, web camera, and/or the like. These and other input devices may connect to processor unit 404 through system bus 402 via interface port(s). Suitable interface port(s) may include, for example, a serial port, a parallel port, a game port, and/or a universal serial bus (USB).

One or more output devices may use some of the same types of ports, and in some cases the same actual ports, as the input device(s). For example, a USB port may be used to provide input to data processing system 400 and to output information from data processing system 400 to an output device. One or more output adapters may be provided for certain output devices (e.g., monitors, speakers, and printers, among others) which require special adapters. Suitable output adapters may include, e.g. video and sound cards that provide a means of connection between the output device and system bus 402. Other devices and/or systems of devices may provide both input and output capabilities, such as remote computer(s) 460. Display 414 may include any suitable human-machine interface or other mechanism configured to display information to a user, e.g., a CRT, LED, or LCD monitor or screen, etc.

Communications unit 410 refers to any suitable hardware and/or software employed to provide for communications with other data processing systems or devices. While communication unit 410 is shown inside data processing system 400, it may in some examples be at least partially external to data processing system 400. Communications unit 410 may include internal and external technologies, e.g., modems (including regular telephone grade modems, cable modems, and DSL modems), ISDN adapters, and/or wired and wireless Ethernet cards, hubs, routers, etc. Data processing system 400 may operate in a networked environment, using logical connections to one or more remote computers 460. A remote computer(s) 460 may include a personal computer (PC), a server, a router, a network PC, a workstation, a microprocessor-based appliance, a peer device, a smart phone, a tablet, another network note, and/or the like. Remote computer(s) 460 typically include many of the elements described relative to data processing system 400. Remote computer(s) 460 may be logically connected to data processing system 400 through a network interface 462 which is connected to data processing system 400 via communications unit 410. Network interface 462 encompasses wired and/or wireless communication networks, such as local-area networks (LAN), wide-area networks (WAN), and cellular networks. LAN technologies may include Fiber Distributed Data Interface (FDDI), Copper Distributed Data Interface (CDDI), Ethernet, Token Ring, and/or the like. WAN technologies include point-to-point links, circuit switching networks (e.g., Integrated Services Digital networks (ISDN) and variations thereon), packet switching networks, and Digital Subscriber Lines (DSL).

Codec 430 may include an encoder, a decoder, or both, comprising hardware, software, or a combination of hardware and software. Codec 430 may include any suitable device and/or software configured to encode, compress, and/or encrypt a data stream or signal for transmission and storage, and to decode the data stream or signal by decoding, decompressing, and/or decrypting the data stream or signal (e.g., for playback or editing of a video). Although codec 430 is depicted as a separate component, codec 430 may be contained or implemented in memory, e.g., non-volatile memory 442.

Non-volatile memory 442 may include read only memory (ROM), programmable ROM (PROM), electrically programmable ROM (EPROM), electrically erasable programmable ROM (EEPROM), flash memory, and/or the like, or any combination of these. Volatile memory 440 may include random access memory (RAM), which may act as external cache memory. RAM may comprise static RAM (SRAM), dynamic RAM (DRAM), synchronous DRAM (SDRAM), double data rate SDRAM (DDR SDRAM), enhanced SDRAM (ESDRAM), and/or the like, or any combination of these.

Instructions for the operating system, applications, and/or programs may be located in storage devices 416, which are in communication with processor unit 404 through system bus 402. In these illustrative examples, the instructions are in a functional form in persistent storage 408. These instructions may be loaded into memory 406 for execution by processor unit 404. Processes of one or more embodiments of the present disclosure may be performed by processor unit 404 using computer-implemented instructions, which may be located in a memory, such as memory 406.

These instructions are referred to as program instructions, program code, computer usable program code, or computer-readable program code executed by a processor in processor unit 404. The program code in the different embodiments may be embodied on different physical or computer-readable storage media, such as memory 406 or persistent storage 408. Program code 418 may be located in a functional form on computer-readable media 420 that is selectively removable and may be loaded onto or transferred to data processing system 400 for execution by processor unit 404. Program code 418 and computer-readable media 420 form computer program product 422 in these examples. In one example, computer-readable media 420 may comprise computer-readable storage media 424 or computer-readable signal media 426.

Computer-readable storage media 424 may include, for example, an optical or magnetic disk that is inserted or placed into a drive or other device that is part of persistent storage 408 for transfer onto a storage device, such as a hard drive, that is part of persistent storage 408. Computer-readable storage media 424 also may take the form of a persistent storage, such as a hard drive, a thumb drive, or a flash memory, that is connected to data processing system 400. In some instances, computer-readable storage media 424 may not be removable from data processing system 400.

In these examples, computer-readable storage media 424 is a non-transitory, physical or tangible storage device used to store program code 418 rather than a medium that propagates or transmits program code 418. Computer-readable storage media 424 is also referred to as a computer-readable tangible storage device or a computer-readable physical storage device. In other words, computer-readable storage media 424 is media that can be touched by a person.

Alternatively, program code 418 may be transferred to data processing system 400, e.g., remotely over a network, using computer-readable signal media 426. Computer-readable signal media 426 may be, for example, a propagated data signal containing program code 418. For example, computer-readable signal media 426 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 418 may be downloaded over a network to persistent storage 408 from another device or data processing system through computer-readable signal media 426 for use within data processing system 400. For instance, program code stored in a computer-readable storage medium in a server data processing system may be downloaded over a network from the server to data processing system 400. The computer providing program code 418 may be a server computer, a client computer, or some other device capable of storing and transmitting program code 418.

In some examples, program code 418 may comprise an operating system (OS) 450. Operating system 450, which may be stored on persistent storage 408, controls and allocates resources of data processing system 400. One or more applications 452 take advantage of the operating system's management of resources via program modules 454, and program data 456 stored on storage devices 416. OS 450 may include any suitable software system configured to manage and expose hardware resources of computer 400 for sharing and use by applications 452. In some examples, OS 450 provides application programming interfaces (APIs) that facilitate connection of different type of hardware and/or provide applications 452 access to hardware and OS services. In some examples, certain applications 452 may provide further services for use by other applications 452, e.g., as is the case with so-called “middleware.” Aspects of present disclosure may be implemented with respect to various operating systems or combinations of operating systems.

The different components illustrated for data processing system 400 are not meant to provide architectural limitations to the manner in which different embodiments may be implemented. One or more embodiments of the present disclosure may be implemented in a data processing system that includes fewer components or includes components in addition to and/or in place of those illustrated for computer 400. Other components shown in FIG. 4 can be varied from the examples depicted. Different embodiments may be implemented using any hardware device or system capable of running program code. As one example, data processing system 400 may include organic components integrated with inorganic components and/or may be comprised entirely of organic components (excluding a human being). For example, a storage device may be comprised of an organic semiconductor.

In some examples, processor unit 404 may take the form of a hardware unit having hardware circuits that are specifically manufactured or configured for a particular use, or to produce a particular outcome or progress. This type of hardware may perform operations without needing program code 418 to be loaded into a memory from a storage device to be configured to perform the operations. For example, processor unit 404 may be a circuit system, an application specific integrated circuit (ASIC), a programmable logic device, or some other suitable type of hardware configured (e.g., preconfigured or reconfigured) to perform a number of operations. With a programmable logic device, for example, the device is configured to perform the number of operations and may be reconfigured at a later time. Examples of programmable logic devices include, a programmable logic array, a field programmable logic array, a field programmable gate array (FPGA), and other suitable hardware devices. With this type of implementation, executable instructions (e.g., program code 418) may be implemented as hardware, e.g., by specifying an FPGA configuration using a hardware description language (HDL) and then using a resulting binary file to (re) configure the FPGA.

In another example, data processing system 400 may be implemented as an FPGA-based (or in some cases ASIC-based), dedicated-purpose set of state machines (e.g., Finite State Machines (FSM)), which may allow critical tasks to be isolated and run on custom hardware. Whereas a processor such as a CPU can be described as a shared-use, general purpose state machine that executes instructions provided to it, FPGA-based state machine(s) are constructed for a special purpose, and may execute hardware-coded logic without sharing resources. Such systems are often utilized for safety-related and mission-critical tasks.

In still another illustrative example, processor unit 404 may be implemented using a combination of processors found in computers and hardware units. Processor unit 404 may have a number of hardware units and a number of processors that are configured to run program code 418. With this depicted example, some of the processes may be implemented in the number of hardware units, while other processes may be implemented in the number of processors.

In another example, system bus 402 may comprise one or more buses, such as a system bus or an input/output bus. Of course, the bus system may be implemented using any suitable type of architecture that provides for a transfer of data between different components or devices attached to the bus system. System bus 402 may include several types of bus structure(s) including memory bus or memory controller, a peripheral bus or external bus, and/or a local bus using any variety of available bus architectures (e.g., Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI)).

Additionally, communications unit 410 may include a number of devices that transmit data, receive data, or both transmit and receive data. Communications unit 410 may be, for example, a modem or a network adapter, two network adapters, or some combination thereof. Further, a memory may be, for example, memory 406, or a cache, such as that found in an interface and memory controller hub that may be present in system bus 402.

### F. Illustrative Embodiments and Claim Concepts

This section describes additional aspects and features of systems and methods for estimating values of water quality target parameters, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

A1. A method for predicting a value of a target parameter based on measurements of surrogate parameters, the method comprising: measuring a first dataset comprising time series data for a plurality of parameters including at least a first surrogate parameter, a second surrogate parameter, a third surrogate parameter, and the target parameter; identifying a plurality of regions in a multidimensional space corresponding to clusters of points of the first dataset in the multidimensional space, wherein the multidimensional space has dimensions corresponding to at least a subset of the plurality of parameters; determining a nonlinear manifold comprising a plurality of modeling functions multiplied by corresponding domain indicator functions, wherein each modeling function models the cluster of points of the first dataset corresponding to a respective one of the regions, and each domain indicator function is defined to be close to one in the region associated with the corresponding modeling function, and close to zero in all of the other regions; measuring a second dataset comprising at least one value of the first surrogate parameter, at least one value of the second surrogate parameter, and at least one value of the third surrogate parameter; and predicting a value of the target parameter corresponding to the second dataset by determining a value of the nonlinear manifold corresponding to the at least one value of the first surrogate parameter, the at least one value of the second surrogate parameter, and the at least one value of the third surrogate parameter.

A2. The method of paragraph A1, wherein the nonlinear manifold passes through a local mean of each cluster of points of the first dataset.

A3. The method of paragraph A1, wherein each domain indicator function comprises a hyperbolic tangent.

A4. The method of paragraph A1, wherein determining the nonlinear manifold comprises obtaining a plurality of fitting parameters of the modeling functions using a nonlinear regression method.

A5. The method of paragraph A4, wherein using the nonlinear regression method includes determining respective sets of initial fitting parameters for each of the modeling functions, such that each modeling function, when evaluated using the respective set of initial fitting parameters, has a value equal to a mean value of the target parameter of the cluster of points corresponding to that modeling function.

A6. The method of paragraph A1, further comprising augmenting a prediction of target parameter value for a first cluster of the plurality of clusters, by: selecting the first cluster from among the plurality of clusters; computing, for the region of the multidimensional space where the first cluster is located, a surface defined by a plurality of radial basis functions, each radial basis function being a function of a local mean value of the first surrogate parameter and a local mean value of a temporal parameter; and predicting a second value of the target parameter, the second value of the target parameter corresponding to a given value of the first surrogate parameter at a time corresponding to a first value of the temporal parameter, by determining a value of the surface corresponding to the given value of the first surrogate parameter and the first value of the temporal parameter.

A7. The method of paragraph A1, wherein the target parameter is a water quality parameter, and the first, second, and third surrogate parameters are discharge rate, turbidity, and chlorophyll respectively.

B1. A method of estimating a target parameter, the method comprising: obtaining values of a plurality of surrogate parameters; determining a nonlinear surface in a phase space defined by the plurality of surrogate parameters, wherein the nonlinear surface defines values of the target parameter; and predicting a first value of the target parameter by identifying a value of the nonlinear surface at a point in the phase space corresponding to the obtained values of the plurality of surrogate parameters.

B2. The method of paragraph B1, wherein determining the nonlinear surface comprises: determining a plurality of local surfaces each configured to model the target parameter in a respective region of the phase space; and joining the plurality of local surfaces together using a plurality of joining functions configured to have a nonzero value at a respective one of the regions and a zero value outside the respective one of the regions.

B3. The method of paragraph B2, wherein determining the plurality of local surfaces comprises: embedding a plurality of known data points in the phase space, each known data point comprising known values of the target parameter and the plurality of surrogate parameters, and each known data point corresponding to a respective time; identifying a plurality of clusters of the known data points in the phase space; defining respective locations of the plurality of clusters in the phase space to be the regions of the phase space; and selecting, as each one of the local surfaces, a respective surface that is linear in the corresponding region of the phase space and passes through a mean of the known values of the target parameter in the corresponding region.

B4. The method of paragraph B3, wherein determining the plurality of local surfaces further comprises using a nonlinear regression to obtain values of constants defining each local surface.

B5. The method of paragraph B3, wherein the obtained values of the plurality of surrogate parameters correspond to a first time that is later than any of the times corresponding to the plurality of known data points.

B6. The method of paragraph B2, wherein each joining function comprises a hyperbolic tangent.

B7. The method of paragraph B1, wherein the target parameter and the surrogate parameters each correspond to water quality parameters.

B8. The method of paragraph B7, wherein the target parameter is a nitrate concentration and the surrogate parameters include at least one of the following: discharge, turbidity, chlorophyll concentration.

B9. The method of paragraph B1, further comprising: obtaining second values of the plurality of surrogate parameters, wherein the obtained second values of the plurality of surrogate parameters correspond to a first region of the phase space; determining a set of values at the first region by subtracting the nonlinear surface at the first region from a mean value of the target parameter with respect to a time parameter and a first one of the surrogate parameters; determining a second surface using a plurality of radial basis functions, such that the second surface approximates the determined set of values in a phase space defined by the time parameter and the first one of the surrogate parameters; and predicting a second value of the target parameter based on a value of the second surface corresponding to the obtained second values of the plurality of surrogate parameters.

B10. The method of paragraph B9, wherein the time parameter is configured to reflect a season of year.

B11. The method of paragraph B10, wherein the time parameter comprises a sinusoidal function having a minimum value at a beginning of a calendar year and a maximum value approximately halfway through the calendar year.

B12. The method of paragraph B1, wherein obtaining the values of the plurality of surrogate parameters comprises measuring the values using one or more sensors disposed in situ at a body of water.

C1. A data processing system, comprising: one or more processors; a memory; and a plurality of instructions stored in the memory and executable by the one or more processors to: receive values of a plurality of surrogate parameters; determine a nonlinear surface in a phase space defined by the plurality of surrogate parameters, wherein the nonlinear surface defines values of a target parameter; and predicting a first value of the target parameter by identifying a value of the nonlinear surface at a point in the phase space corresponding to the received values of the plurality of surrogate parameters.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A method for predicting a value of a target parameter based on measurements of surrogate parameters, the method comprising:
measuring a first dataset comprising time series data for a plurality of parameters including at least a first surrogate parameter, a second surrogate parameter, a third surrogate parameter, and the target parameter;
identifying a plurality of regions in a multidimensional space corresponding to clusters of points of the first dataset in the multidimensional space, wherein the multidimensional space has dimensions corresponding to at least a subset of the plurality of parameters;
determining a nonlinear manifold comprising a plurality of modeling functions multiplied by corresponding domain indicator functions, wherein each modeling function models the cluster of points of the first dataset corresponding to a respective one of the regions, and each domain indicator function is defined to be close to one in the region associated with the corresponding modeling function, and close to zero in all of the other regions;
measuring a second dataset comprising at least one value of the first surrogate parameter, at least one value of the second surrogate parameter, and at least one value of the third surrogate parameter; and
predicting a value of the target parameter corresponding to the second dataset by determining a value of the nonlinear manifold corresponding to the at least one value of the first surrogate parameter, the at least one value of the second surrogate parameter, and the at least one value of the third surrogate parameter.

2. The method of claim 1, wherein the nonlinear manifold passes through a local mean of each cluster of points of the first dataset.

3. The method of claim 1, wherein each domain indicator function comprises a hyperbolic tangent.

4. The method of claim 1, wherein determining the nonlinear manifold comprises obtaining a plurality of fitting parameters of the modeling functions using a nonlinear regression method.

5. The method of claim 4, wherein using the nonlinear regression method includes determining respective sets of initial fitting parameters for each of the modeling functions, such that each modeling function, when evaluated using the respective set of initial fitting parameters, has a value equal to a mean value of the target parameter of the cluster of points corresponding to that modeling function.

6. The method of claim 1, further comprising augmenting a prediction of target parameter value for a first cluster of the plurality of clusters, by:
selecting the first cluster from among the plurality of clusters;
computing, for the region of the multidimensional space where the first cluster is located, a surface defined by a plurality of radial basis functions, each radial basis function being a function of a local mean value of the first surrogate parameter and a local mean value of a temporal parameter; and
predicting a second value of the target parameter, the second value of the target parameter corresponding to a given value of the first surrogate parameter at a time corresponding to a first value of the temporal parameter, by determining a value of the surface corresponding to the given value of the first surrogate parameter and the first value of the temporal parameter.

7. The method of claim 1, wherein the target parameter is a water quality parameter, and the first, second, and third surrogate parameters are discharge rate, turbidity, and chlorophyll respectively.

8. A method of estimating a target parameter, the method comprising:
obtaining values of a plurality of surrogate parameters;
determining a nonlinear surface in a phase space defined by the plurality of surrogate parameters, wherein the nonlinear surface defines values of the target parameter; and
predicting a first value of the target parameter by identifying a value of the nonlinear surface at a point in the phase space corresponding to the obtained values of the plurality of surrogate parameters.

9. The method of claim 8, wherein determining the nonlinear surface comprises:
determining a plurality of local surfaces each configured to model the target parameter in a respective region of the phase space; and
joining the plurality of local surfaces together using a plurality of joining functions configured to have a nonzero value at a respective one of the regions and a zero value outside the respective one of the regions.

10. The method of claim 9, wherein determining the plurality of local surfaces comprises:
embedding a plurality of known data points in the phase space, each known data point comprising known values of the target parameter and the plurality of surrogate parameters, and each known data point corresponding to a respective time;
identifying a plurality of clusters of the known data points in the phase space;
defining respective locations of the plurality of clusters in the phase space to be the regions of the phase space; and
selecting, as each one of the local surfaces, a respective surface that is linear in the corresponding region of the phase space and passes through a mean of the known values of the target parameter in the corresponding region.

11. The method of claim 10, wherein determining the plurality of local surfaces further comprises using a nonlinear regression to obtain values of constants defining each local surface.

12. The method of claim 10, wherein the obtained values of the plurality of surrogate parameters correspond to a first time that is later than any of the times corresponding to the plurality of known data points.

13. The method of claim 9, wherein each joining function comprises a hyperbolic tangent.

14. The method of claim 8, wherein the target parameter and the surrogate parameters each correspond to water quality parameters.

15. The method of claim 14, wherein the target parameter is a nitrate concentration and the surrogate parameters include at least one of the following:
discharge, turbidity, chlorophyll concentration.

16. The method of claim 8, further comprising:
obtaining second values of the plurality of surrogate parameters, wherein the obtained second values of the plurality of surrogate parameters correspond to a first region of the phase space;
determining a set of values at the first region by subtracting the nonlinear surface at the first region from a mean value of the target parameter with respect to a time parameter and a first one of the surrogate parameters;
determining a second surface using a plurality of radial basis functions, such that the second surface approximates the determined set of values in a phase space defined by the time parameter and the first one of the surrogate parameters; and
predicting a second value of the target parameter based on a value of the second surface corresponding to the obtained second values of the plurality of surrogate parameters.

17. The method of claim 16, wherein the time parameter is configured to reflect a season of year.

18. The method of claim 17, wherein the time parameter comprises a sinusoidal function having a minimum value at a beginning of a calendar year and a maximum value approximately halfway through the calendar year.

19. The method of claim 8, wherein obtaining the values of the plurality of surrogate parameters comprises measuring the values using one or more sensors disposed in situ at a body of water.

20. A data processing system, comprising:
one or more processors;
a memory; and
a plurality of instructions stored in the memory and executable by the one or more processors to:
receive values of a plurality of surrogate parameters;
determine a nonlinear surface in a phase space defined by the plurality of surrogate parameters, wherein the nonlinear surface defines values of a target parameter; and
predicting a first value of the target parameter by identifying a value of the nonlinear surface at a point in the phase space corresponding to the received values of the plurality of surrogate parameters.
